(19) United States
(12) Patent Application Publication
(10) Pub. No.: US 2021/0381971 A1
(43) Pub. Date: Dec. 9, 2021

(54) DEHAZE AND BACTERIOSTATIC FILM

(71) Applicant: Cheng-Sheng Tsung, Nantou County (TW)

(72) Inventor: Cheng-Sheng Tsung, Nantou County (TW)

(21) Appl. No.: 16/894,313

(22) Filed: Jun. 5, 2020

(51) Int. Cl. G01N 21/552; G01N 21/65

(52) U.S. Cl. CPC G01N 21/554; G01N 21/658

(57) ABSTRACT

A dehaze and bacteriostatic film includes a substrate material layer and a composite surface plasmon layer formed on the substrate material layer. The composite surface plasmon layer includes a particle stacked film layer and a particle suspension layer located on the substrate material layer, and the particle stacked film layer and the particle suspension layer jointly generate a composite surface plasmon wave. Accordingly, the composite surface plasmon wave is excited by visible light, so that different types of surface plasmon waves generated by the structures resonate and multiply with each other. The different surface plasmon waves add up to generate electromagnetic field intensity capable of dissociating spatial materials at a certain distance, such as water vapor to be partially ionized which is rich in hydroxide ions with effects of dehazing and inhibiting growth of bacteria.

### FIELD OF THE INVENTION

The invention relates to a dehaze and bacteriostatic structure, and more particularly to a dehaze and bacteriostatic structure excited by visible light.

### BACKGROUND OF THE INVENTION

Medical institutions, libraries, schools, indoor playgrounds, public transportation systems, and other indoor places or closed spaces are hotbeds for germs due to the large number of people entering and leaving. For the need of public health, regular disinfection to dehaze and inhibit growth of bacteria is a necessary measure.

As to the conventional methods for dehaze and bacteriostasis, bacteriostasis methods are quite diverse which are roughly divided into a normal sterilization method used locally and an unusual sterilization method needed clearance, the removal of people and appliance. For example, the former method uses bacteriostatic materials to make items, hand sanitizers (alcohol), masks, and so on; and the latter method use of spraying disinfectant water in localized areas, use of photocatalyst with ultraviolet or with strong excitant deep-ultraviolet for disinfection, and so on.

However, said methods cause the problem that bacteriostatic materials will gradually lose effectiveness over time, hand sanitizers are not mandatory and have the concern of harming the skin, use of spraying disinfectant water causes a bad smell problem, and use of photocatalyst with ultraviolet or with strong excitant deep-ultraviolet for disinfection need to remove people to prevent organisms damaged and need to remove appliance damaged by ultraviolet.

As for haze removal, it is common to use dehaze equipment, such as indoor air purifiers and outdoor dehaze towers, etc., but they consume a lot of resources, and causes invisible pollution when manufacturing these appliances, which is even more harmful.

As for portable dehaze method, wearing a mask causes discomfort, bringing along a portable negative ionizer affects physical activity, and equipping a car with an air cleaner not only occupies space, but also causes ozone hazard. Most of the dehaze equipment need to be regularly replaced with consumables, and thus have been criticized by the public for the high costs of use. Moreover, it is known that bacteriostasis relates to inhibiting the growth of microorganisms, and most of the method for dehaze use filter filtering or electrostatic adsorption. Thus, only a few ways can have both functions of bacteriostasis and dehaze at the same time. However, the methods that provide both functions of bacteriostasis and dehaze are applied in modular or electrical mode (nanoe, plasmacluster); in addition to high carbon footprints, huge amounts of energy are consumed and huge amounts of consumables are produced. Obviously, it is difficult for the conventional dehaze and bacteriostasis method to meet the requirements in usage.

### SUMMARY OF THE INVENTION

A main object of the present invention is to disclose a dehaze and bacteriostatic structure which has both bacteriostasis and dehaze efficacies and can be used at all times.

A secondary object of the present invention is to disclose a dehaze and bacteriostatic structure that does not need to consume huge amounts of energy and produce huge amounts of consumables, has low carbon emissions, and can be used indoors and outdoors at all times with minimum restrictions.

In order to achieve the above objects, the present invention provides a dehaze and bacteriostatic film comprising a substrate material layer and a composite surface plasmon layer, wherein the composite surface plasmon layer is formed on the substrate material layer. The composite surface plasmon layer includes a particle stacked film layer and a particle suspension layer. The particle stacked film layer and the particle suspension layer jointly generate a composite surface plasmon wave.

Accordingly, the present invention provides a dehaze and bacteriostatic film, which is excited by visible light to form the composite surface plasmon wave. Accordingly, different types of surface plasmon waves generated by the structures resonate and multiply with each other. The different surface plasmon waves add up to generate electromagnetic field intensity capable of dissociating spatial materials at a certain distance, such as water vapor to be partially ionized which is rich in hydroxide ions, and thereby producing effects of dehazing and inhibiting growth of bacteria in a surrounding environment through continuous generation of hydroxide ions. Further, the effects of dehazing and inhibiting growth of bacteria can be enhanced by increasing a thickness (number of layers) of the particle stacked film layer and the particle suspension layer to provide for using in other fields with different requirements.

### BRIEF DESCRIPTION OF THE DRAWINGS

FIG. 1 is a cross-sectional view of a structure of a first embodiment of a dehaze and bacteriostatic film of the invention;

FIG. 2 is a microscopic cross-sectional view of a structure of the first embodiment of the dehaze and bacteriostatic film of the invention;

FIG. 3 is a microscopic cross-sectional view of a structure of a second embodiment of a dehaze and bacteriostatic film of the invention;

FIG. 4 is a microscopic cross-sectional view of a structure of a third embodiment of a dehaze and bacteriostatic film of the invention;

FIG. 5 is a microscopic cross-sectional view of a structure of a fourth embodiment of a dehaze and bacteriostatic film of the invention;

FIG. 6 is a microscopic cross-sectional view of a structure of a fifth embodiment of a dehaze and bacteriostatic film of the invention;

FIG. 7 is a microscopic cross-sectional view of a structure of a sixth embodiment of a dehaze and bacteriostatic film of the invention; and

FIG. 8 is a microscopic cross-sectional view of a structure of a seventh embodiment of a dehaze and bacteriostatic film of the invention.

### DETAILED DESCRIPTION OF THE PREFERRED EMBODIMENTS

The detailed description and technical contents of the present invention are described below with reference to the drawings.

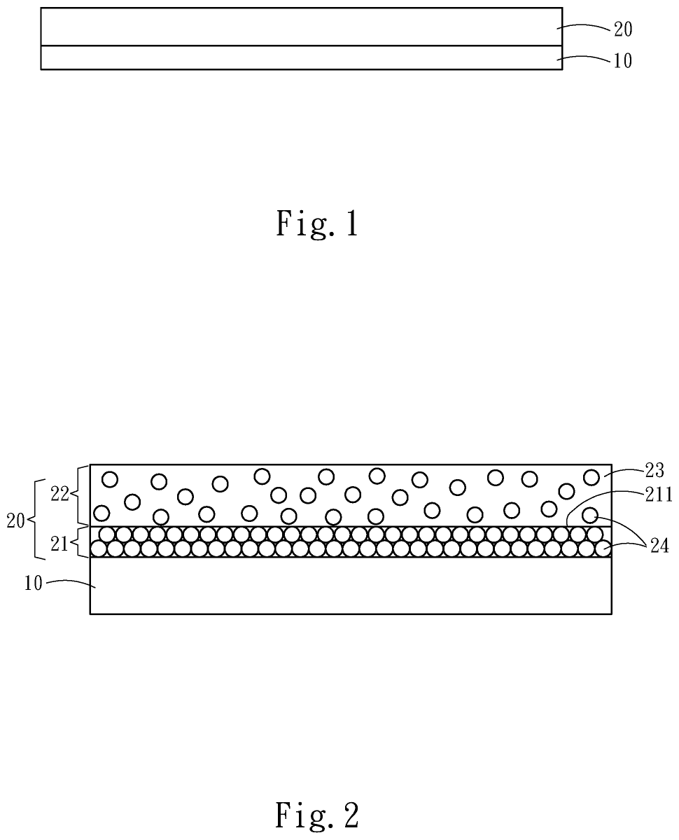

Please refer to FIG. 1 and FIG. 2 for a first embodiment. As shown in the figures, the present invention is a dehaze and bacteriostatic film comprising a substrate material layer 10 and a composite surface plasmon layer 20. The composite surface plasmon layer 20 is formed on the substrate material layer 10.

The composite surface plasmon layer 20 includes a particle stacked film layer 21 and a particle suspension layer 22. In detail, the particle stacked film layer 21 is located on the substrate material layer 10, and the particle suspension layer 22 is located on the particle stacked film layer 21.

Furthermore, a surface 211 of the particle stacked film layer 21 away from the substrate material layer 10 releases a plurality of unsteady-state nanoparticles 24. The plurality of unsteady-state nanoparticles 24 are composed of nanoparticles selected from a group of metals, metal compounds and metal mixtures. For example, metal materials are such as copper, platinum, aluminum, or mixtures of the foregoing metals with a particle size between 1 nm and 100 nm; or compounds, alloys, or mixtures of the foregoing metal materials. A dielectric carrier layer 23 is provided on the particle stacked film layer 21. The particle suspension layer 22 is formed by infiltration or diffusion of the plurality of unsteady-state nanoparticles 24 into the dielectric carrier layer 23 during a manufacturing process. In detail, the plurality of unsteady-state nanoparticles 24 enters the dielectric carrier layer 23 in a chemical or physical manner, such as infiltration, diffusion, etc., to form the particle suspension layer 22 to generate localized surface plasmon resonance (LSPR).

With the structures described above, the composite surface plasmon layer 20 generates a composite surface plasmon wave. In one embodiment, the substrate material layer 10 is a dielectric material and a light-transmitting material. The choice of material and whether the substrate material layer 10 is transparent can be decided based on requirements of usage.

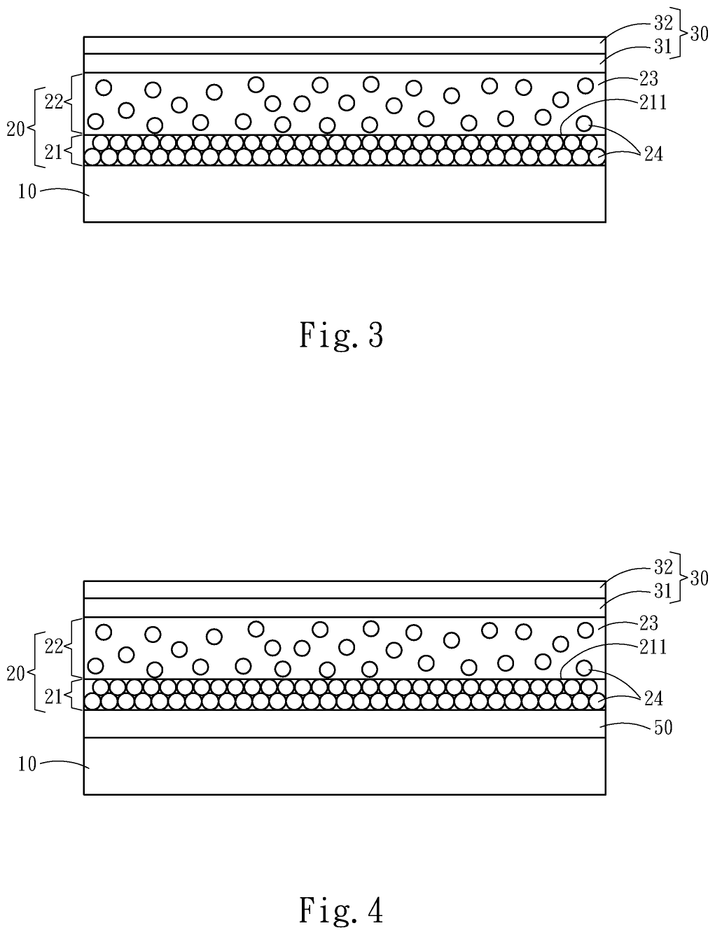

Please refer to FIG. 3 for a second embodiment of the present invention. Compared with the first embodiment, a functional layer 30 is further formed on the particle suspension layer 22. The functional layer 30 is capable of providing functions including adhesion, tearing off and sticking, protection, anti-scratch, self-cleaning, electric conduction (soft ITO conductive layer on solar cell or display), anti-fog, and so on. The functional layer 30 is produced by chemical and physical methods such as immersion, roll coating, blade coating, adhesion, spray coating, vapor deposition, sputtering, and chemical vapor deposition. Different manufacturing processes are used for different requirements of usage. For example, the functional layer 30 with an adhesive effect is made by roll coating, blade coating, or spray coating. It can be adhered on a glass or a flat surface as a glass heat-insulating film, which includes both dehaze and bacteriostasis effects. The functional layer 30, made by hardened and stacked, has high anti-scratch and wear-resistance functions and also includes dehaze and bacteriostasis effects. The functional layer 30 with transparent conductive effect is formed by roll coating, blade coating, spray coating, vapor deposition, sputtering and chemical vapor deposition. And it can be used in touch panels or flexible displays with both dehaze and bacteriostasis effects.

As shown in FIG. 3, the functional layer 30 includes an adhesive layer 31 and a release layer 32. The adhesive layer 31 is formed on the particle suspension layer 22. The adhesive layer 31 can be formed on the particle suspension layer 22 by roll coating, or by blade coating. The release layer 32 covers the adhesive layer 31. In one embodiment, the release layer 32 is a release film, which is a film with a separative surface. Then production methods for the release film include, but are not limited to, polyethylene terephthalate (PET), polyethylene (PE), or o-phenylphenol (OPP) processed films to be treated by plasma, coated with fluorine, or coated with a silicone release agent.

As shown in FIG. 2, further, the particle stacked film layer 21 is formed on the substrate material layer 10 by common thin film manufacturing methods such as spray coating, immersion, blade coating, roll coating, adsorption, and spin coating. If immersion is used as a manufacturing process, in order to help adsorption or enhance the effect, substances not limited to nano size (including a size larger than nano size), such as material particles of metals, non-metals, chemical compounds and mixtures can be added to a solution to help adsorption or enhance the effect.

In the present invention, the spin coating method is used as an example. A nano-structured metal is easily charged to control concentration, rotation speed and baking temperature to form the particle stacked film layer 21 on the substrate material layer 10. In this way, a thickness and an arrangement mode of the particle stacked film layer 21 can be controlled. Also, because of the spin coating method, nano metal particles of the particle stacked film layer 21 are not arranged regularly. After spin coating and drying, the surface 211 of the particle stacked film layer 21 away from the substrate material layer 10 is capable of releasing the plurality of unsteady-state nanoparticles 24. Finally, the dielectric carrier layer 23 is formed on the surface 211. If the dielectric carrier layer 23 is formed by chemical and physical methods of roll coating, blade coating, spray coating, vapor deposition, sputtering, adhesion, adsorption, spin coating, and chemical vapor deposition, the plurality of unsteady-state nanoparticles 24 will enter the dielectric carrier layer 23 by chemical or physical method of infiltration or diffusion to form the particle suspension layer 22.

Accordingly, the particle stacked film layer 21 is formed by stacking the unsteady-state nanoparticles 24. A surface energy of the unsteady-state nanoparticles 24 is large, thereby the particle stacked film layer 21 is formed on the substrate material layer 10 by naturally stacking the unsteady-state nanoparticles 24, and because the unsteady-state nanoparticles 24 are suspended and distributed in the particle suspension layer 22, a cross-linked three-dimensional structure is formed. Therefore, a large number of two-dimensional planar film layers of the unsteady-state nanoparticles 24 contacting with each other are continuously stacked, and the particle suspension layer 22 simultaneously generates a large number of two-dimensional planar film layers of the unsteady-state nanoparticles 24 that are not in contact with each other.

Therefore, the particle stacked film layer 21 generates N surface plasmon resonances (SPR) as a layered structure with a limited thickness, and the particle suspension layer 22 simultaneously generates N localized surface plasmon resonances (LSPR) as a non-layered structure. Therefore, the particle stacked film layer 21 and the particle suspension layer 22 jointly generate a composite surface plasmon wave.

As shown in FIG. 3, gluing is to coat the particle suspension layer 22 (the dielectric carrier layer 23) with an adhesive to form the adhesive layer 31, and filming is to allow the release layer 32 to cover the adhesive layer 31 to be adhered on the particle suspension layer 22 (the dielectric carrier layer 23). The release layer 32 is a release film used to protect and shield the adhesive layer 31 and preserve the adhesion function, while protecting the composite surface plasmon layer 20; however, if the dielectric carrier layer 23 (the particle suspension layer 22) with scratch resistance is selected, of course, the release film may not be selected to use.

When using the dehaze and bacteriostatic film of the present invention, as long as simply tear off the release layer 32, the adhesive layer 31 can directly stick to appropriate positions such as windows, lamp holders, automobile glass, mobile phone screens, etc. Visible light excites the composite surface plasmon wave in partially ionized air, such as water vapor to be partially ionized which is rich in hydroxide ions (OH—), and producing a bactericidal effect similar to nanoe. In detail, the composite surface plasmon wave produces a waterfall-like effect to impact substances with wave motions, causing partial substances to be ionized and to carry out positive and negative electricity due to energy absorption. Further, water vapor and oxygen with positive and negative electricity will inhibit growth of bacteria and decompose dirt. Similarly, this method also causes suspended particles to carry out positive and negative electricity and self aggregated, resulting in effects of dehaze and bacteriostasis.

Further, in the present invention, water vapor is ionized to be rich in hydroxide ions (OH—). Considering the generated OH— ions are coated in water molecules or water molecule groups, it is not easily to be reduced or eliminated by the environment, and thereby more partially ionized molecules can be produced and moving farther, so that the effects of dehaze and bacteriostasis can fill an entire space or open area.

Please refer to FIG. 4 for a third embodiment of the present invention. Compared with the first embodiment, a functional dielectric layer 50 is disposed between the particle stacked film layer 21 and the substrate material layer 10. The functional dielectric layer 50 includes modified surface functions of enhancing adsorbability, flatness, hydrophilicity, hydrophobicity, heat resistance, and acid resistance, or group functions of the above, or also includes functions such as adhesion, electric conduction, scratch resistance, wear resistance, static adsorption, repeated tearing off and sticking, and anti-fouling or anti-fog, or group functions of the above.

And the invention can choose whether to form the functional layer 30 on the particle suspension layer 22. The functional layer 30 is located above the functional dielectric layer 50, and the functional layer 30 includes the adhesive layer 31 and the release layer 32. The adhesive layer 31 is formed on the particle suspension layer 22, and the release layer 32 covers the adhesive layer 31. In this embodiment, functions of the functional dielectric layer 50 can be used to increase adhesion, reduce hydrophobicity, and so on.

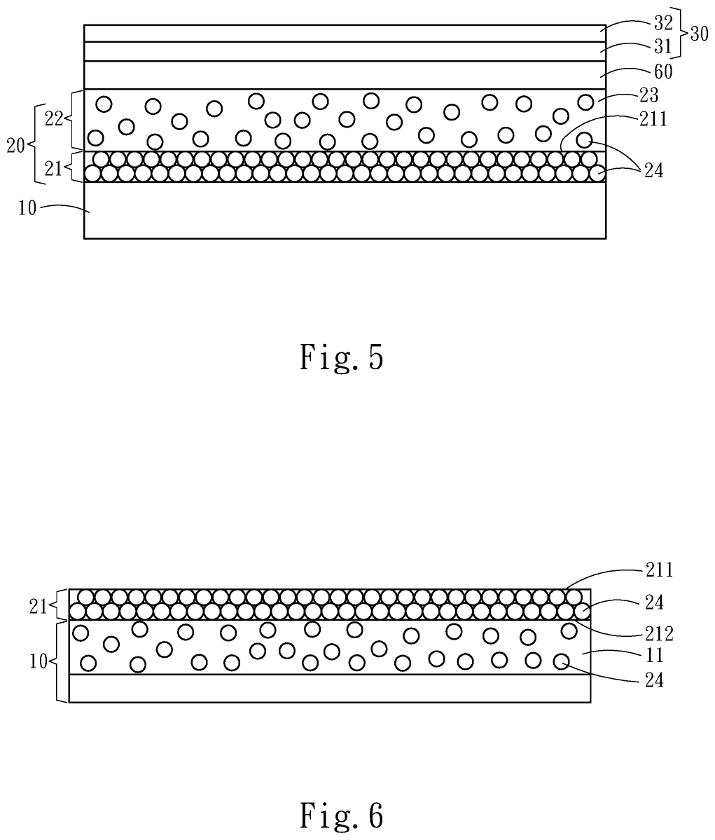

Please refer to FIG. 5 for a fourth embodiment of the present invention. Compared with the first embodiment, the fourth embodiment further includes a functional dielectric layer 60 located on the particle suspension layer 22. The functional dielectric layer includes functions of improving adsorbability, flatness, hydrophilicity, hydrophobicity, heat resistance, and acid resistance, or group functions of the above, or also includes functions such as adhesion, electric conduction, scratch resistance, wear resistance, static adsorption, repeated tearing off and sticking, and anti-fouling or anti-fog, or group functions of the above. And the invention can choose whether to form the functional layer 30 on the functional dielectric layer 60. The functional layer 30 further includes the adhesive layer 31 and the release layer 32. The adhesive layer 31 is formed on the functional dielectric layer 60, and the release layer 32 covers the adhesive layer 31.

In addition to the functional dielectric layer 60 being used to increase adhesion, reduce hydrophobicity, etc., the functional dielectric layer 60 is also used as the dielectric carrier layer 23 to form the particle suspension layer 22.

Please refer to FIG. 6 for a fifth embodiment of the present invention. A surface 212 of the particle stacked film layer 21 adjacent to the substrate material layer 10 releases the plurality of unsteady-state nanoparticles 24. The unsteady-state nanoparticles 24 infiltrate or diffuse into the substrate material layer 10 in a chemical or physical manner to form an additional particle suspension layer 11. In a manufacturing process, the particle stacked film layer 21 is formed on the substrate material layer 10 by spraying, immersion, blade coating, roll coating, adsorption, spin coating, etc., or formed under environments such as high heat, high pressure, vacuum, etc., and the plurality of unsteady-state nanoparticles 24 of the particle stacked film layer 21 infiltrate or diffuse into the substrate material layer 10 chemically or physically, and form the additional particle suspension layer 11 jointly with the substrate material layer 10.

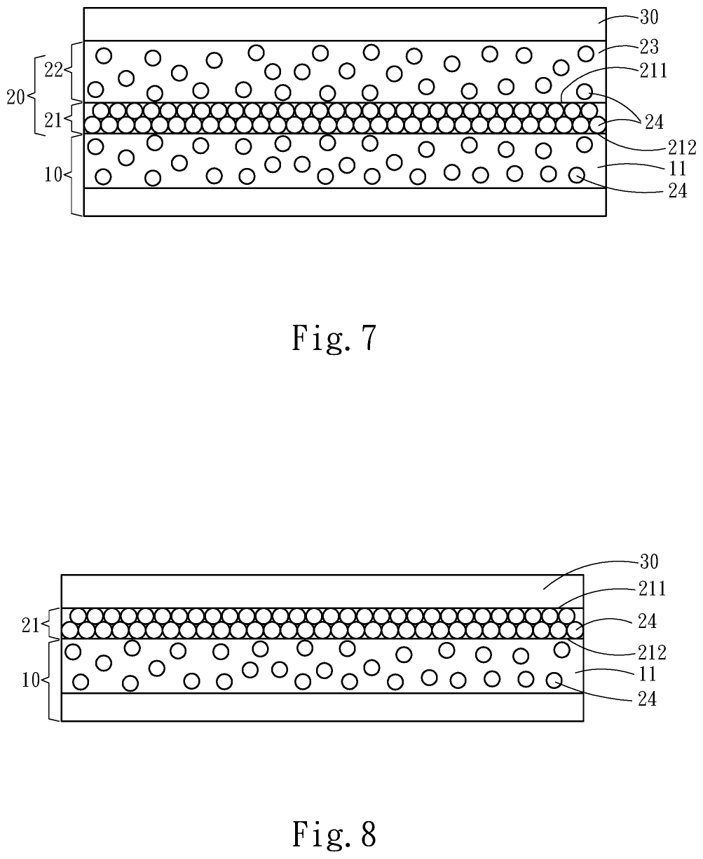

Please refer to FIG. 7 for a sixth embodiment of the present invention. Compared with the fifth embodiment, a functional dielectric carrier layer 23 is further disposed on the particle stacked film layer 21. Both the surface 211 and the surface 212 of the particle stacked film layer 21 release the plurality of unsteady-state nanoparticles 24. The unsteady-state nanoparticles 24 infiltrate or diffuse into the substrate material layer 10 and the functional dielectric carrier layer 23 in a chemical or physical manner to form the additional particle suspension layer 11 and the particle suspension layer 22. Functions of the functional dielectric carrier layer 23 include adhesion, tearing off and sticking, protection, scratch resistance, self-cleaning, electric conduction (soft ITO conductive layer on solar cell or display), anti-fog, and so on. Furthermore, the particle suspension layer 22 is formed with the functional layer 30, and functions of the functional layer 30 are as described above, and will not be repeated.

Please refer to FIG. 8 for a seventh embodiment of the present invention. Only the surface 212 of the particle stacked film layer 21 adjacent to the substrate material layer 10 releases the plurality of unsteady-state nanoparticles 24, and the unsteady-state nanoparticles 24 infiltrate or diffuse into the substrate material layer 10 in a chemical or physical manner to form the additional particle suspension layer 11. Compared with the fifth embodiment, the functional layer 30 is formed on the particle stacked film layer 21. Functions of the functional layer 30 are as described above, and will not be repeated.

In summary, features of the present invention are:

1. To take visible light as an excitation light source, it doesn't need clearance to meet the requirement of being effective 24 hours a day.

2. When being used, there is no bad smell, no environmental toxicity, and no harmful substances are produced, which can meet the requirements of public health.

3. It can be used in various places for a long period of time, effectively dehazing and inhibiting growth of bacteria, and maintaining public health and safety.

4. No consumables that need to be replaced regularly or with certain quantity or amount, and thus will not cause secondary pollution.

5. It can be used indoors, outdoors, in transportation, etc., with minimum restrictions in usage.

6. It provides for long-term use without recession period.

What is claimed is:

1. A dehaze and bacteriostatic film comprising:
a substrate material layer; and
a composite surface plasmon layer, formed on the substrate material layer, the composite surface plasmon layer including a particle stacked film layer and a particle suspension layer, wherein the particle stacked film layer and the particle suspension layer jointly generate a composite surface plasmon wave.

2. The dehaze and bacteriostatic film as claimed in claim 1, wherein a dielectric carrier layer is provided on the particle stacked film layer, a surface of the particle stacked film layer away from the substrate material layer releases a plurality of unsteady-state nanoparticles, and the plurality of unsteady-state nanoparticles enters the dielectric carrier layer through either infiltration or diffusion to form the particle suspension layer.

3. The dehaze and bacteriostatic film as claimed in claim 2, wherein a functional layer is further formed on the particle suspension layer.

4. The dehaze and bacteriostatic film as claimed in claim 3, wherein a functional dielectric layer is further disposed between the particle stacked film layer and the substrate material layer.

5. The dehaze and bacteriostatic film as claimed in claim 3, wherein a functional dielectric layer is further located on the particle suspension layer, and the functional layer is located on the functional dielectric layer.

6. The dehaze and bacteriostatic film as claimed in claim 2, wherein a functional dielectric layer is further disposed between the particle stacked film layer and the substrate material layer.

7. The dehaze and bacteriostatic film as claimed in claim 2, wherein a functional dielectric layer is further located on the particle suspension layer.

8. The dehaze and bacteriostatic film as claimed in claim 1, wherein a surface of the particle stacked film layer adjacent to the substrate material layer releases a plurality of unsteady-state nanoparticles, and the plurality of unsteady-state nanoparticles enters the substrate material layer through either infiltration or diffusion to form the particle suspension layer.

9. The dehaze and bacteriostatic film as claimed in claim 8, wherein a functional dielectric carrier layer is further formed on the particle stacked film layer.

10. The dehaze and bacteriostatic film as claimed in claim 9, wherein a surface of the particle stacked film layer away from the substrate material layer releases a plurality of unsteady-state nanoparticles, and the plurality of unsteady-state nanoparticles enters the functional dielectric carrier layer through either infiltration or diffusion to form an additional particle suspension layer.

11. The dehaze and bacteriostatic film as claimed in claim 10, wherein a functional layer is formed on the additional particle suspension layer.

12. The dehaze and bacteriostatic film as claimed in claim 8, wherein a functional layer is further formed on the particle stacked film layer.
